(19) United States
(12) Patent Application Publication
(10) Pub. No.: US 2004/0133144 A1
(43) Pub. Date: Jul. 8, 2004

(54) Waterproof animal cast and bandage protector

(76) Inventor: Michael P. Crichton, Orlando, FL (US)

Correspondence Address: Michael P. Crichton, 12015 Shadowbrook Ln., Orlando, FL, 32828, US

(21) Appl. No.: 10/336,504

(22) Filed: Jan. 3, 2003

(51) Int. Cl. A61F 13/00

(52) U.S. Cl. 602/62

(57) ABSTRACT

Waterproof animal cast and bandage protector for keeping dry and clean an animal's injured limb, which has been covered by a cast or bandage. An elongated tube with two ends, one open end, where the animal's limb is inserted and one closed end. The elongated tube is made of a durable and stretchable covering being smaller in diameter than the cast or bandage, allowing it to conform to the casts or bandages shape. The open end opens via a flexible ring, being smaller in diameter than the cast or bandage. That ring creates a seal and holds tight when cover is unrolled over the animal's cast or bandage. The closed end has a non-skid surface to protect from tearing and to help prevent slipping when the animal is on a potentially slippery surface.

This invention relates to cast and bandage protectors, in particular to a simple covering that allows a pet owner to quickly and reliably cover their pet's cast or bandage and allows the animal to walk on rough surfaces without tearing the covering and walk on potentially slippery surfaces without slipping, and claims the benefit of priority to U.S. Provisional Patent Application titled Waterproof Animal Cast and Bandage Protector filed Jan. 2, 2002.

### BACKGROUND AND PRIOR ART

Similar to humans, an animal's cast or bandages must be kept dry and clean. Unlike humans, most animals/pets must venture outdoors every day. When venturing outdoors it is very likely that the cast or bandage will become wet or dirty. In order to prevent that from happening the pet owner must somehow cover the cast or bandage to keep it dry and clean. Without said invention, the pet owner must go through the tedious and unreliable task of finding a waterproof material (usually some kind of plastic wrap), finding some way to secure it to the pet (usually by tape or rubber bands) and keeping the pet still long enough to secure the covering. In addition to being tedious and frustrating, the above is also very unreliable. Since the animal walks on the above manufactured covering it is often torn or comes off all together, resulting in a wet or dirty cast or bandage.

Over the years many inventions have been proposed for humans but none specifically for animals. See for example U.S. Pat. No. 4,139,003 filed on Apr. 4, 1977. Though these past inventions are adequate for covering casts and bandages when bathing, none of them address the specific needs of animals. An animal coving must address specific needs. It must be strong enough for the animal to walk on different types of surfaces without tearing or coming off. It must also be able to stand up to an animal's claws without tearing. The covering must also have a non-skid type surface as to help prevent the animal from slipping on potentially slippery surfaces. Lastly the covering must have a reliable seal at the top in order to keep water from coming in and to keep the entire covering from coming off all together. The most similar human cast cover U.S. Pat. No. 4,139,003 although similar, is not meant to be walked on, is not intended for animals and does not address the specific needs of animals.

### SUMMARY OF THE INVENTION

The primary objective of the invention is to provide a quick, simple and effective way for animal owners to cover and keep clean and dry their animal's injured limb, which has been covered by a cast or bandage. As compared to conventional covering means such as plastic wrap or bags sealed by tape or rubber bands this invention will save the user/pet owner a lot of time and frustration, while covering the animal's cast or bandage much more effectively. The invention eliminates the frustrating, tedious and usually unsuccessful task of wrapping and sealing the animal's injured limb every time the animal must venture outdoors. The unique feature of this invention is that it is designed specifically for animals. Unlike human cast covers that are designed to keep a cast dry while bathing or swimming, this invention addresses a completely different need in that it is designed so that the animal can walk on the cover while being worn without their claws 4 breaking through it. Another one of this invention's unique features is the non-skid surface which helps prevent slipping or skidding when the animal is walking on potentially slippery surfaces. The special animal design of this cast and bandage cover is what makes it so unique as compared to any human cast cover.

The invention is an elongated tube with two ends, one open end 1, where the dog's paw is inserted and one closed end 2. The invention is made of a durable and stretchable covering being smaller in diameter than the cast or bandage, allowing it to conform to the casts or bandages shape. The invention is made with a “sealing” ring 3 at the open end. When relaxed, FIG. 1 the flexible ring is smaller in diameter than the dog's paw. When stretched, unrolled and fitted over the dog's entire cast or bandage FIG. 4 the flexible ring holds firmly against the dog's leg, creating a seal, preventing moisture or dirt from contacting the cast or bandage.

The invention is made to keep dry and clean a dog's injured limb, which has been covered by a cast or bandage. Due to the extreme variation in the size of dogs, their limbs and the casts or bandages that are placed on them, the invention is designed to seal at a varying range of lengths. The invention is used as follows: While making sure the non-skid surface is under the dog pads, the user/dog owner stretches the cover over the dog's paw and cast or bandage. The user then proceeds to unroll the cover, much like a condom is unrolled. Once the cover has been unrolled to the point at which the cast or bandage has been completely covered, the tight ring creates a seal preventing moisture or dirt from affecting the cast or bandage. When the dog returns in doors the user/dog owner simply unrolls the invention and removes it dog's paw.

### BRIEF DESCRIPTION OF THE FIGURES

FIG. 1 is a perspective view of the invention in its relaxed position, before it has been stretched over the dog's cast or bandage;

FIG. 2 is a view of the invention as it is starting to be stretched over and unrolled up and over the dog's paw, approaching the cast 6 or bandage;

FIG. 3 is a view of the invention as it is stretched over and unrolled up most of the dog's cast or bandage;

FIG. 4 is a view of the invention as it is stretched over and unrolled up the entire cast or bandage, where the elastic portion above the cast creates a seal;

FIG. 5 is a bottom view of the invention as to show the non-skid surface that the animal walks on;

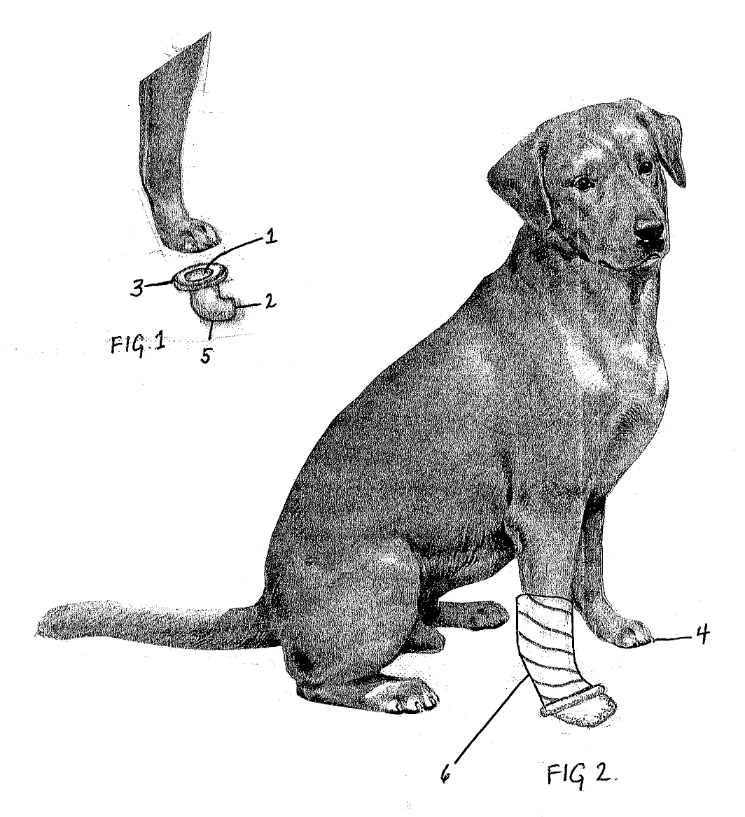

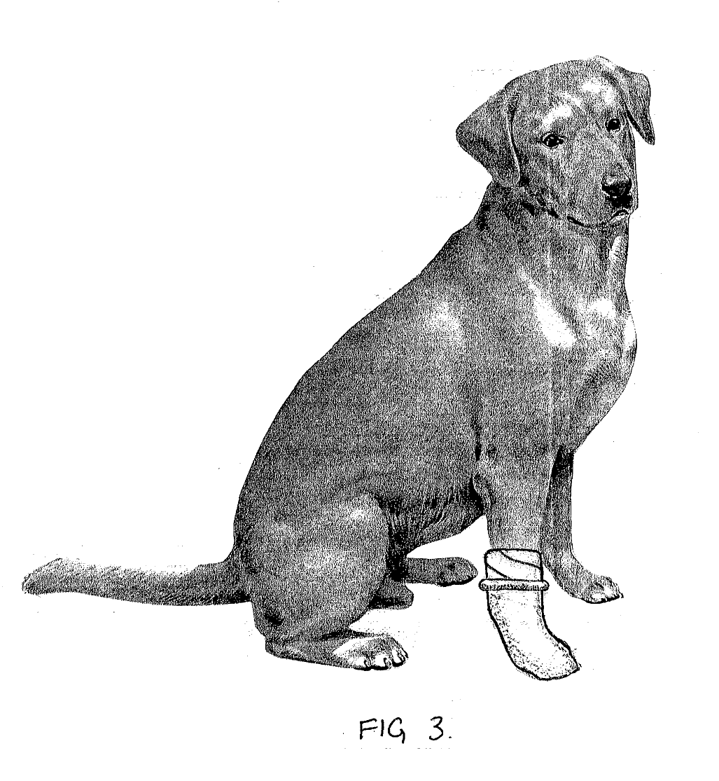

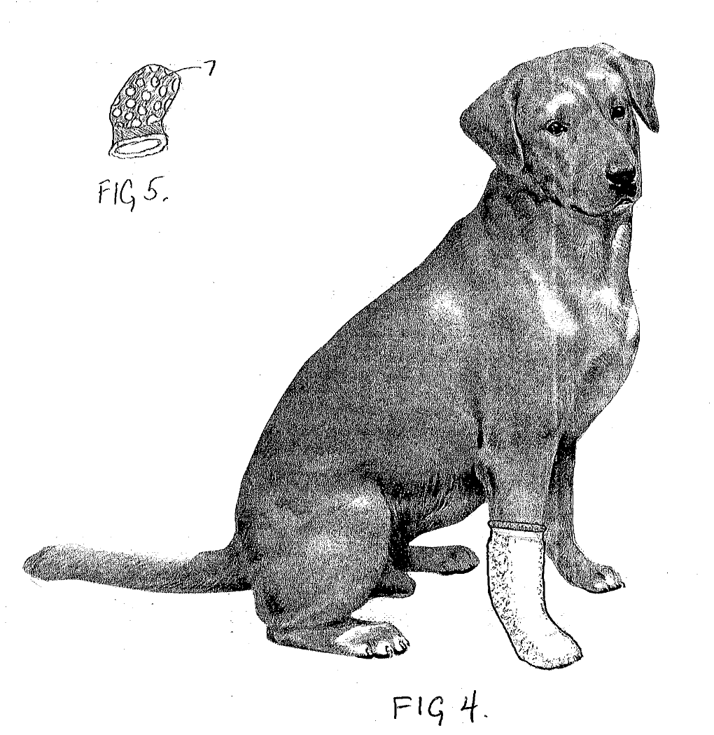

What I claim is:

1. An animal cast and bandage protector, which is a flexible and stretchable and is or has:
stretched over the cast or bandage then extended and unrolled up the animal's limb until over the cast or bandage;
a stretchable elongated tube having two ends;
a flexible and stretchable, open end to accept animal limbs or different sizes and respective casts or bandages of different sizes;
an open end with a flexible and stretchable, yet durable ring, being thicker and smaller in diameter than the elongated tube, to help hold and create a seal when unrolled over the whole cast or bandage;
a closed end, made of a heavy material intended to withstand the demands (potential puncturing) an animal's claws would place on said material when walking on a variety of rough and dirty surfaces;
a closed end, made with a non-skid design to help prevent slipping or sliding when the animal is walking on wet and potentially slippery surfaces;
an elongated tube that, when not stretched over the animal's cast or bandage, is smaller in diameter than the cast or bandage that it is being placed over;
an elongated tube that, when stretched over the animal's cast or bandage, keeps the covered portion of the limb and the said cast or bandage clean and dry whether the animal is walking in snow, rain, mud or being bathed;
